(19) United States
(12) Patent Application Publication
(10) Pub. No.: US 2015/0182157 A1
(43) Pub. Date: Jul. 2, 2015

(54) On-Patient Autonomous Blood Sampler and Analyte Measurement Device

(71) Applicants: Varun Boriah, Union City, CA (US); Beelee Chua, Seoul (KR); Ramesh Damani, Sunnyvale, CA (US)

(72) Inventors: Varun Boriah, Union City, CA (US); Beelee Chua, Seoul (KR); Ramesh Damani, Sunnyvale, CA (US)

(73) Assignee: CardioCanary, Inc., Union City, CA (US)

(21) Appl. No.: 14/143,918

(22) Filed: Dec. 30, 2013

(51) Int. Cl. A61B 5/15; A61B 5/1477; A61B 5/157; A61B 5/1455; G06F 19/00; A61B 5/151

(52) U.S. Cl. CPC A61B 5/150755; G06F 19/3418; A61B 5/15109; A61B 5/157; A61B 5/150969; A61B 5/150343; A61B 5/15087; A61B 5/1455; A61B 5/1477

(57) ABSTRACT

The invention relates to systems, apparati, and methods for real-time diagnostics to detect and diagnose disease conditions in patients. In an embodiment, the apparatus is attached to a patient, takes samples of the patient's blood, allows real-time detection of markers in the patient's blood, and provides rapid diagnosis of the patient.

### FIELD OF THE INVENTION

The invention relates to systems, apparati, and methods for real-time diagnostics to detect and diagnose disease conditions in patients.

### BACKGROUND OF THE INVENTION

Every minute of every hour of every day, an American dies of heart attack. Myocardial Infarction (MI) or heart attack is one of the leading causes of death in the United States.

Each year 8 MM patients in the US (15 MM worldwide) are admitted to emergency rooms (ER) for chest pain. For a very small fraction of these patients, diagnosis of MI is easily accomplished based on their ECG and appropriate treatment is provided to these patients in a timely manner. For the vast majority of the rest of these patients, the challenge is to rapidly triage them to determine which 10-15% need immediate intervention and which can be safely discharged.

Early detection and diagnosis of an MI has long been pursued by doctors, clinicians and researchers alike in order to reduce mortality rates and minimize longer term complications due to heart tissue necrosis. In recent years, the identification of cardiac biomarkers and in particular cardiac troponin has provided clinicians with a highly specific tool to reliably detect an MI. This is especially significant for cases where electrocardiograms (ECGs) are non-diagnostic. Recent guidelines issued jointly by AHA/ACC dictate a series of blood tests (serial measurement) in order to establish the elevated and rapidly changing cardiac troponin concentration required for an accurate diagnosis. Current emergency department protocols call for initial testing upon patient admission, with subsequent testing between 4 and 6 hours and finally between 8 and 12 hours. During the hours it takes to confirm the clinical diagnosis of MI, heart tissue necrosis continues, patient prognosis worsens, and 30-day mortality rates can reach nearly 15%. Additionally, holding patients in the ER for the hours it takes to complete the protracted sequence of tests contributes greatly to ER overcrowding and increased healthcare costs.

Recent groundbreaking clinical data has shown that by increasing the frequency of testing, a diagnosis of MI can be made significantly sooner. This approach, termed accelerated serial measurement, will allow doctors to initiate anti-ischemics, anti-thrombolytics, or surgical intervention much sooner with significantly improved short-term and long-term prognoses for the patient. The benefits of accelerated serial measurement are significantly amplified by the use of high-sensitivity troponin assays. By lowering the concentration of troponin that can be measured with appropriate precision, the high sensitivity assays can detect smaller changes in troponin concentrations sooner and enable more rapid rule-in/rule-out decisions for acute myocardial infarction. These sensitive assays also permit improved and rapid risk stratification of patients.

Unfortunately, accelerated serial measurement has not been adopted in emergency departments due to their inability to perform higher frequency testing using existing protocols, personnel and equipment (central laboratory and point-of-care instruments). The average turn-around-time for each test executed by a nurse is nearly 3 hours (blood draw to data review by the lab director to test results delivered to emergency doctor), even though running the actual assay takes under 10 minutes. Today, less than 3% of lab-based troponin test results meet the ACC/AHA guideline for a 30 minute turnaround time. Given that every year over 8 million Americans are admitted to the emergency department with complaints of chest pain, accelerated serial measurement is impossible to implement without automating the blood sampling, measurement and data reporting process.

Several attempts within the diagnostics industry to develop bedside systems to automate the blood sampling, measurement and data reporting process have been unsuccessful and have not gained traction. These large and bulky systems occupied precious space and were cumbersome to use within emergency departments. They also required an ER nurse to collect blood samples periodically (very challenging in an ER department where staffing is limited and chaos is the norm) and had to be plugged into a wall power socket, severely restricting patient mobility. The solution to address all these shortcomings is to develop a miniaturized, automated sample collection, testing, and reporting system that is completely self-contained, allows patients to be ambulatory, and can be operated with minimal intervention by ER staff.

### SUMMARY OF THE INVENTION

The invention relates to systems, apparati, and methods for real-time diagnostics to detect and diagnose disease conditions. In a preferred embodiment, the disease condition is a Myocardial Infarction. An embodiment of the present invention describes an apparatus for real-time detection of cardiac markers in patients.

In an embodiment, the apparatus comprises a means of penetrating the skin, extracting a blood sample, a means for introducing the blood sample to a biosensor fluidic circuit, a means for analysis of the sample, and a means for reporting the result.

In one embodiment, the apparatus comprises a sensor with a means for collecting a biological sample. In a preferred embodiment, the sensor is an immunosensor and the biological sample is blood. In an embodiment, the means for collecting the biological sample is a skin puncturing needle that is spring loaded and controlled by an electromechanical actuator that activates the skin puncturing needle at preset times. In an embodiment, the apparatus also comprises a means for wireless data communication for receiving signals and transmitting signals. In a preferred embodiment, the means for wireless data communication receives an authentication signal from the hospital electronic record systems and transmits data to the same. An embodiment of the apparatus also includes an electronic display for showing status of the measurement, immunosensor data, and/or patient data. An embodiment of the apparatus comprises an on board computer for controlling the immunosensor, the means for wireless data communication, and the electromechanical actuator of the skin puncturing needle or needle array and electromechanical actuators for deploying stored reagents.

In a preferred embodiment, the apparatus comprises a cardiac marker electrochemical-based immunosensor module with individual spring-loaded skin puncturing needles and a vent-free expandable waste reservoir; an electromechanical actuator to release the spring-loaded skin puncturing needles at programmed intervals; a mechanism for introducing reagents into the immunosensor module; a wireless data transceiver that receives an authentication signal from a hospital electronic record and sends processed immunosensor data; at least one diagnosis algorithm that uses the immunosensor data to perform a rapid diagnosis of myocardial infarction; an electronic display showing a status of measurement, the immunosensor data as well as a patient data; and an onboard computer that controls release of the skin puncturing needles, operation of the immunosensor, data processing, data transmission, and the electronic display.

In an embodiment of the invention, the apparatus comprises at least two sensor modules for making measurements on biological sample(s) at different time points. In another embodiment the measurements on biological sample(s) can be made at the same time point. In another embodiment the measurements on the biological sample(s) may be for the same analyte or different analytes. In a preferred embodiment, the apparatus has three sensors each of which is an immunosensor for detecting troponin levels in the patient's blood.

In an embodiment of the invention, the apparatus also comprises a temperature sensor which records temperature data. In a preferred embodiment, the temperature data is used by the on board computer to compensate for temperature fluctuations in analyzing the data from the sensors.

In an embodiment of the invention, the apparatus also comprises an alarm for identifying a patient adverse event. In a preferred embodiment, the apparatus comprises at least one audible and one visual alarm.

In an embodiment, the apparatus comprises an electrochemical sensor electrode and a related method of fabrication that enables a significant increase in the surface area of the electrode when compared to conventional silicon electrodes of the same footprint. The three dimensional sensing electrodes are silicon-based metal sputtered electrochemical electrodes. The increase in surface area may be two-fold to several hundred-fold.

The larger surface area is made possible by etching a series of vertical trenches into the silicon substrate. Trenches may be created using a number of processes, including but not limited to mechanical dicing, photo-patterning and ion etching, etc. Variability in surface area is minimized due to the precision of the manufacturing processes.

The silicon substrate may be sputtered with gold, platinum, or any other appropriate electrically conductive material to create electrodes.

The invention also relates to methods for real-time detection and diagnosis of disease conditions. In a preferred embodiment, the disease condition detected and diagnosed is a Myocardial Infarction. In an embodiment, the method comprises attaching the apparatus to the patient's body preferably via a skin adhesive, entering patient data into the apparatus, communicating the patient data to the hospital electronic records and receiving an authentication signal. On authentication, a spring-loaded skin puncturing needle is released to puncture the skin at a pre-programmed time and the blood sample is allowed to collect at the site of skin penetration until a minimum volume is generated. The blood sample is transported through microfluidic channels into a sensor chamber with an electrochemical-based cardiac marker immunosensor via capillary wicking. Immunoassay reagents are automatically introduced into the sensor chamber and the excess blood sample and reagents are collected in a waste chamber. The cardiac marker concentration is measured via an electrochemical signal. The data is made available locally via a display on the apparatus and transmitted to hospital electronic records. The apparatus uses algorithms to aid in the diagnosis of a Myocardial Infarction based on the cardiac marker data. The testing may be repeated at a pre-programmed interval or run on an ad-hoc basis.

In a preferred embodiment, the method for real-time detection of myocardial infarction comprises the steps of contacting an apparatus to a patient's skin, wherein the apparatus comprises an onboard computer, a wireless transceiver, a skin puncturing needle, a sample chamber, an immunosensor module, and a vent-free expandable waste reservoir; inputting a patient information into the on board computer; receiving an authentication signal at the apparatus from a first hospital electronic record via the wireless transceiver; releasing the skin puncturing needle at a programmed time to puncture the skin; collecting a blood sample from the punctured skin in the sample chamber of the apparatus; transporting the blood sample from the sample chamber into the immunosensor module after a predetermined volume of blood is collected; deploying an immunoassay reagent in the immunosensor module and routing an excess of the blood sample into the vent-free expandable waste reservoir; measuring the cardiac marker concentration in the immunosensor module; transmitting an immunosensor data to a second hospital electronic record; and running at least one diagnosis algorithm that uses the immunosensor data to perform a rapid diagnosis of myocardial infarction.

The invention also relates to a method of fabrication that enables a significant increase in the surface area of the electrode when compared to conventional silicon electrodes of the same footprint. Three dimensional sensing electrodes are silicon-based metal sputtered electrochemical electrodes with larger surface area than conventional silicon electrodes of the same footprint. The increase in surface area may be two-fold to several hundred-fold. The larger surface area is made possible by etching a series of vertical trenches into the silicon substrate. Trenches may be created using a number of processes, including but not limited to mechanical dicing, photo patterning and ion etching, etc. Variability in surface area is minimized due to the precision of the manufacturing processes. The silicon substrate may be sputtered with gold, platinum, or any other appropriate electrically conductive material to create electrodes.

It is a goal of the invention to shift the clinical practice paradigm through the automation of the entire process of diagnostics: biological sample acquisition through testing all the way to result communication. For example, multiple tests for cardiac troponin, or any other blood analyte, can be run at pre-determined frequencies using a single disposable cartridge. The patient-centric solution can drive a 10-fold improvement in test-order-to-test-result turnaround time. This streamlined system could ensure that patient diagnosis is not delayed due to a lack of resources. For example, a diagnosis (rule-in/rule-out) of chest pain can be completed 4 times faster as compared to the current standard of care (6-12 hours). Expedited triaging will lead to reduced emergency room wait times, superior clinical outcomes, increased hospital operational efficiency, and lower overall healthcare costs. Emergency room personnel can be freed to focus on more critical patients.

A goal of the present invention is to reduce the burden on hospital staff and free them to focus on other critical tasks as well as to spend more quality time with patients and their families. Patients can easily be moved from the emergency department to an alternate in-hospital setting without disrupting the testing process. The potentially catastrophic consequences of blood sample mix-up or mislabeling are altogether eliminated. The system also minimizes the volume of blood drawn from the patient for each test which is of great importance for anemic and critically ill patients. Since our invention is self-contained, the device may be deployed in the field and utilized in ambulances, with test results being available before the patient reaches the hospital.

It is a goal of the invention to also enable simplified serial testing of a variety of clinically relevant biomarkers or analytes found in blood such as glucose, electrolytes, lactate, blood gases, etc.

### DESCRIPTION OF THE DRAWINGS

FIG. 1 illustrates the top view of the architecture of a disposable cartridge.

FIG. 2 illustrates the side view of the architecture of an individual assay unit within the disposable cartridge.

FIG. 3 illustrates the architecture of the electrochemical sensor comparing the Three Dimensional Sensing (3D-Sensing) architecture with a conventional silicon electrode. FIG. 3A illustrates a side view of the electrodes, and FIG. 3B illustrates a top view of the electrodes.

FIG. 4 shows the schematic of a catheter-disposable cartridge docked on the reusable portion.

FIG. 5 shows the catheter-disposable cartridge.

FIG. 6 shows the actuator layout of the reusable portion for the catheter-disposable cartridge.

FIG. 7 illustrates the side view of the architecture of an alternate embodiment with the individual assay unit connected to the disposable cartridge.

FIG. 8 shows the exterior of an embodiment of the assay unit and disposable cartridge.

FIG. 9 shows the interior of an embodiment of the assay unit and disposable cartridge.

### DETAILED DESCRIPTION OF THE INVENTION

In one embodiment, the apparatus of the invention is a medical device about the size of a credit card and ¾ inches thick. It is to be used for chest pain patients suspected of having myocardial infarction or heart attack. It is attached to a chest pain patient's skin. In an embodiment, the apparatus is attached the patient's skin via a skin adhesive. At programmed intervals, the apparatus obtains a biological sample that is placed in contact with a sensor. The sensor measures the cardiac marker(s) concentration and communicates to the hospital electronic system. In an embodiment, the apparatus is able to make at least 3 serial measurements, with each measurement using a fresh biological sample and a fresh sensor. Using at least 3 serial measurement data, the doctors can diagnose the chest pain patient more rapidly.

In a preferred embodiment, the apparatus automatically samples blood from a patient at fixed or programmable intervals and analyzes the blood sample for target proteins/biomarkers, blood gases, electrolytes and/or other blood analytes. The apparatus allows for the collection and analysis of a plurality of blood samples. Patient's blood work can be performed with a minimum of intervention from the hospital staff. A method of the invention comprises collection of samples of patient's blood, a means of analysis of said sample of blood, and reporting the results of said sample analysis.

The apparatus is worn on the patient's body. In one embodiment, the device is worn on the patient's arm. In another embodiment, the device is worn on the arm, the thigh, the back, the abdomen, or any other suitable area. The apparatus may be attached to the body through the use of adhesives, bands, overlays, straps, etc.

In an embodiment, the analysis results are transmitted to the hospital electronic records system (Electronic Medical Records, Laboratory Information Systems, etc) for viewing and real-time diagnoses of the results. The results may also be made available locally on the apparatus or on a visual display in communication with the apparatus.

In an embodiment, the apparatus consists of a reusable portion and a disposable cartridge. The disposable cartridge is adhered or attached to the patient's body and the reusable portion is docked onto it in order to achieve electrical and mechanical communication/connection. In another embodiment, the reusable portion of the apparatus is adhered or attached to the patient's body and the catheter-disposable cartridge is docked or attached to the reusable portion.

The reusable portion may consist of mechanisms, electronics, wireless transmission technology, actuators, displays, etc. In an embodiment, the reusable portion consists of, but is not limited to, a set of miniature actuators, electronics for control, software for sensor operation, data acquisition and data transmission, electrical connectors, a means for data entry, a temperature sensor, a means of temperature regulation, batteries and a display. Most components that do not come in direct contact with the patient's blood reside in the reusable portion.

In an embodiment, the disposable cartridge comprises all or most of the components that come into contact with the patient's blood including but not limited to the piercing element, piercing mechanism, biosensor fluidic circuit, mechanism for generating vacuum or suction, means of moving fluid through the biosensor fluidic circuit, reservoir for collecting waste fluids, chemical reagents, biological reagents, buffers, reaction solutions, and electrodes.

In an embodiment, the disposable cartridge comprises a means to allow adequate blood to collect at the site of skin penetration prior to transporting it through the microfluidic channels to avoid the risk of entrapping air. This is achieved by integrating a capillary force valve into the microfluidic channel. A capillary force valve is a fluid control structure that uses superficial tension at the interface between two immiscible fluids (air/liquid) to block and/or restore the entrance of fluids in microfluidic channels filled with the second fluid (air). The capillary force valve may be created by altering the surface energy of the microfluidic channel through a hydrophobic path or through changing the geometric shape of the microfluidic channel though a sudden expansion.

In an embodiment, the catheter-disposable cartridge consists of tubing and fluidic connectors for blood sample acquisition, an array of biosensors, flushing liquid reservoirs and waste liquid reservoirs, multiple layers of fluidic circuits, diaphragm/membrane valves, flow constrictions, flow expansions, electrical connectors. All components that come in direct contact with the patient's blood reside in the catheter-disposable cartridge. It may also consist of a means of connecting to an intravenous line/catheter/access site.

In another embodiment, the disposable cartridge houses of an array of Individual Assay Units. Each Individual Assay Unit further consists of a means of blood sample acquisition, a means of transporting the blood sample to a biosensor, a means of running an immunoassay, and a means of storing the waste solution. Specifically, the Individual Assay Unit consists of a lancing array, a lancing mechanism, a blood sampling chamber, multiple layers of microfluidic circuits, reagent reservoirs, a waste chamber, a biosensor, electrical contacts, flow constrictions, flow expanders, fluid sensors, and temperature sensors. Further, the biosensor may consist of electrochemical electrodes, and immobilized primary and secondary antibodies. The disposable cartridge may also have a means of attaching to the patient's body, a means of mechanically connecting to the reusable portion and a means of regulating temperature. In an embodiment, all components that come in direct contact with the patient's blood reside in the disposable cartridge.

The microfluidics of the apparatus may be constructed using silicon micromachining, glass micromachining, plastic micromolding, or laminate stack techniques.

In an embodiment, the disposable cartridge is docked/connected to the reusable portion in order to achieve electrical and mechanical communication/connection. When activated, pumps, actuators, electronics and software housed in the reusable portion interface with the disposable cartridge in order to draw a sample of blood from the body and introduce it into an individual sensor chamber or an array of targeted sensor chambers. Once the blood sample has been introduced into the sensor chamber, the biosensor housed in the disposable cartridge along with the components housed in the reusable cartridge measures the concentration of the specific analyte.

The disposable cartridge may contain an array of biosensors that are all capable of performing a single assay or may be capable of performing many different assays for different analytes. The disposable cartridge may contain the means to perform one of more assays within a single biosensor chamber

When activated, actuators, electronics, and software housed in the reusable portion interface with the disposable cartridge in order to draw a sample of blood from the body. In an embodiment, once the blood sample has been obtained, it is introduced into a sensor chamber and the rest of the constituents of the device are used to measure the concentration of the analyte of interest. One or more analytes of interest can be measured using each blood sample. Multiple measurements of the same analyte may be made from a single blood sample.

In an embodiment, penetration of the skin is achieved by a self-contained mechanism that includes a piercing element, a means of puncturing the skin using the piercing element and further may consist of a means of retracting the piercing element out of the skin. The depth of penetration of the skin is sufficient to induce bleeding at site of skin penetration. The piercing element may remain in the skin through the process of blood sample extraction or may be removed prior to blood sample extraction or removed during the process of blood sample extraction.

The piercing element may include, but is not limited to, a hypodermic needle, a blade, a lancet, a solid needle, a hollow needle. The piercing element may consist of a single unit or several individual units or an array of multiple units.

A mechanism for puncturing the skin using the piercing element may include a spring-loaded mechanism, shape memory alloy actuator, solenoid actuator, foam or rubber or compliant disks. The mechanism may be housed in either the disposable or reusable portion.

A means of retracting the piercing element out of the skin may include a spring-loaded mechanism, solenoid actuator, foam or rubber or compliant disks, shape memory alloy actuator. The mechanism may be housed in either the disposable or reusable portion.

In another embodiment, patient's blood may be accessed through an intravenous catheter/cannula, venipuncture, PICC, peripheral catheter/cannula, arterial line/catheter.

In an embodiment, a sampling chamber surrounds the site of skin penetration. The sampling chamber is designed to prevent any leak of the blood sample or intrusion of air into the blood sample at the site of skin penetration. The volume of the sampling chamber may be minimized in order to minimize the risk of excessive bleeding.

The blood sample may be extracted from the site of skin penetration passively via blood pressure, through the application of a vacuum, application of pressure, through the use of capillary forces, any combination thereof, or any equivalent means.

In another embodiment, the blood sample may be extracted from the blood access passively via blood pressure, though the application of vacuum, or any combination thereof. The vacuum may be generated through the use of a pump including but not limited to a peristaltic pump, a syringe pump, and a diaphragm pump.

In an embodiment, the system may consist of a means to manipulate the area surrounding the site of skin penetration in order to locally increase the blood flow and in turn increase the amount of blood extracted. This may consist of the application of pressure, vibration, heat, etc.

The blood sample may be transported from the sampling chamber through the biosensor fluidic circuit through the application of vacuum or pressure or through the use of capillary forces, or any combination thereof.

The extraction of the blood sample and the transport of the blood sample through the biosensor fluidic circuit may be achieved through independent steps. This would allow for independent control of the extraction step from the transport step and would also enable different flow parameters for each step.

In another embodiment, the blood sample may be transported from the blood access through the biosensor fluidic circuit passively via blood pressure, through the application of vacuum, or any combination thereof. The vacuum may be generated through the use of a pump including but not limited to a peristaltic pump, a syringe pump, and a diaphragm pump.

In an embodiment, the vacuum to extract the blood sample may be created by the use of a compliant bag or any alternate means. The vacuum created by the compliant bag ensures that the blood sample moves through the biosensor fluidic circuit at a controlled flow rate. The controlled expansion of the vacuum bag may be achieved through the use of actuators, springs, and mechanisms housed either within the reusable cartridge or the disposable portion or straddling across both portions. The compliant bag may be a self-restoring bag. The compliant bag, used to create the vacuum, may also serve as a waste fluid reservoir.

In an embodiment, the use of capillary forces to extract and transport the blood sample may be aided through the use of selective coatings and the use of appropriate channel dimensions. The coating may include but not limited to hydrophilic and hydrophobic coatings.

In an embodiment, the biosensor fluidic circuit may consist of a constriction to minimize the extent of reaction solution that enters the sampling chamber from the biosensor fluidic circuit.

In an embodiment, the biosensor fluidic circuit may contain a means to detect the presence of fluid at various points of the fluidic path. The fluid may be sensed through the use of detectors including but not limited to electrodes, electrochemical electrodes, optical sensors placed along the fluid path.

The biosensor fluidic circuit may consist of a means to temporarily restrict the fluid flow at various points of the fluidic path. Temporary restrictions may be created through the use of active or passive valves. One embodiment of a passive valve may be a capillary valve. The capillary valve may be created by a sudden change in the fluid path dimensions or through varying the surface energy of the fluid path.

The device may contain features to filter out cells from the blood sample. This can be achieved using features engineered into the biosensor fluidic circuit or an in line filter or any other similar suitable means.

The biosensor fluidic circuit may contain an antibody-enzyme conjugate, an area where a capture antibody is immobilized with respect to the flow channels, electrochemical electrodes and one or more pouches containing solutions. The electrochemical sensor may further comprise enzymes, proteins, polymer coatings or deposits.

The antibody-enzyme conjugate and the capture antibody may be in the form of dried reagent in the fluid flow path of the biosensor fluidic circuit. The blood sample may be drawn into the biosensor fluidic circuit through the application of vacuum or pressure or through the use of capillary forces or any combination thereof, and flows past the antibody-enzyme conjugate. The antibody-enzyme conjugate dissolves into the blood sample and the antibody-enzyme conjugate binds to the target molecule in the blood sample.

In an embodiment, the continued application of vacuum or pressure or through the use of capillary forces or any combination thereof, the blood sample with the antibody-enzyme conjugate continues toward the area where the target molecule bound by the antibody-enzyme conjugate is captured and bound to immobilized capture antibody.

Solution is released to flush away the blood sample, unbound target molecule, and untethered antibody-enzyme conjugate. The solution in the pouch may be discharged through the use of actuators, springs, mechanisms housed either within the reusable or the disposable or straddling across both portions. Solutions may be released concurrently or sequentially. The deployment of solutions may be used to transport the blood through the biosensor fluidic circuit and toward the waste reservoir.

Alternately, a separate pouch may contain solution with reagents to react with the antibody-enzyme conjugate. The solution in the pouch may be discharged through the use of actuators, springs, mechanisms housed either within the reusable or the disposable or straddling across both portions.

Air may be introduced into the biosensor fluidic circuit in order to flush away the blood sample, unbound target molecule, and untethered antibody-enzyme conjugate prior to introduction of the reagents that react with the enzyme.

Pressure and vacuum cycles may be used to facilitate mixing of the reaction constituents as well as the transport of the reaction constituents through the biosensor fluidic circuit. The pressure and vacuum cycles may be generated through the use of the compliant bag.

The biosensor of the invention can detect cardiac markers such as cardiac troponin (I and T), myoglobin, CK-MB, copeptin, B-type natriuretic peptide (BNP), N-terminal fragment B-type natriuretic peptide (NT-proBNP), C-reactive proteins (CRP), amongst others. The biosensor of the invention may also detect other analytes of interest including glucose, electrolytes (sodium, potassium, magnesium, calcium, etc), blood gases (oxygen and carbon dioxide), and metabolites (creatinine, urea, nitrogen), cholesterol, markers of sepsis (lactate and procalcitonin), stroke, heart failure, clotting, among others. Additional analytes that may be detected by the biosensor include metal ions, proteins, enzymes, antibodies, sugars, hormones, drugs, carbohydrates, amongst others.

In an embodiment, the biosensor may be based on an electrochemical detection schema with electrochemical electrodes. Alternatively, the biosensor can use optical detection methods and apparati well known in the art. Other tests which are well-known in the art for measuring the target molecule or a target molecule conjugate may be used in the invention. For example, the invention may detect the target molecule or products from an assay detecting the target molecule using fluorescent labels, optical absorbance, label-free quantification, chemiluminescence, color-changing, surface acoustic waves, amperometric, coulometric, field effect transistor charge transfer from protein binding process, 2-electrode electrochemical detection, 3-electrode electrochemical detection, optical detection, electrical detection, magnetic detection, carbon nanotubes electrodes, silicon electrodes, screen printed electrodes, ceramic electrodes, resonance detection, electromagnetic wave absorbance, electromagnetic wave emission, etc.

In one embodiment, reagents in the solution react with the enzyme of the target molecule::antibody-enzyme conjugate, and the reaction product is measured by the electrochemical electrodes. The measured value is recorded and the corresponding target molecule concentration is deduced and transmitted to the hospital electronic record.

Variations to the immunoassay and detection schema include an ELISA with enzyme labels, an ELISA with fluorescent labels, optical absorbance, label-free quantification, chemiluminescence, color-changing, surface acoustic waves, amperometric, coulometric, field effect transistor charge transfer from protein binding process, 2-electrode electrochemical detection, 3-electrode electrochemical detection, optical detection, electrical detection, magnetic detection, carbon nanotubes electrodes, silicon electrodes, screen printed electrodes, ceramic electrodes, resonance detection, electromagnetic wave absorbance, electromagnetic wave emission, etc.

The device may further comprise bubble detectors, blood detectors, fluid detectors, temperature sensors, air vents, red blood cell filters, and/or debubblers. These may be housed in either the reusable portion or the disposable cartridge.

The apparatus may also comprise a means of measuring the temperature within the disposable cartridge, within the reaction solution pouches, and/or within the biosensor fluidic circuit. Further the system may consist of a means to vary the temperature within the disposable cartridge, within the reaction solution pouches, and/or within the biosensor fluidic circuit.

The disposable cartridge may contain a multitude of biosensor assemblies or assay units. The biosensor assemblies or assay units housed in the disposable cartridge may be configured to detect the same analyte or different analytes. These include, but are not limited to, blood gases, electrolytes, lactate, glucose, proteins, biomarkers, etc.

The system may have multiple biosensors or assay units to allow sequential testing at multiple time points using a single system. Each biosensor or assay unit may be used for single measurement or may be used for multiple measurements. The sampling and measurement cycle can be repeated using a preprogrammed routine by using a fresh biosensor assembly within the disposable cartridge for each subsequent measurement. The sampling and measurement sequence may be repeated in an adjustable routine.

In one embodiment, each assay unit may be individually packaged, separate from the disposable cartridge. In use, the assay unit is unpacked and docked into a cavity in the disposable cartridge. This allows the user to select the appropriate set of assay units for a specific patient. Also, malfunctioning assay units may be replaced with a new assay unit without discarding the entire disposable cartridge.

At the end of measurement cycle, the disposable cartridge is disposed and the reusable portion is recovered for cleaning and re-use.

Algorithms useful in the invention are known in the art. In an embodiment, the algorithm extracts the difference of cardiac marker concentration between measurements and compares it against a known benchmark to determine whether the patient is undergoing a myocardial infarction. In another embodiment, the algorithm compares cardiac marker concentration against known cutoff values to determine whether the patient is undergoing a myocardial infarction. In another embodiment, the algorithm compares the 1^{st} measurement against a known benchmark and then calculates the next measurement time point where the 2^{nd} blood sampling and measurement will take place based on the first measurement. It can also subsequently use the 1^{st} and 2^{nd} measurements to determine the measurement time point for the 3^{rd} measurement in order to extract the most relevant change in the cardiac marker concentration to determine whether the patient is undergoing a myocardial infarction. In another embodiment, the algorithm generates trend lines to indicate the increase or decrease in cardiac marker concentration and the rate of change of the cardiac marker concentration. In another embodiment, an algorithm can forewarn hospital staff if the generated trend line indicates the patient will eventually cross the benchmark/threshold value. In another embodiment, the algorithm processes the series of cardiac marker measurements and generates a graphical display of the data. In another embodiment, the algorithm compares the cardiac marker measurements to a look-up table to aid clinicians with risk stratification of patients.

The apparatus may communicate using any wireless transmission protocol such as Bluetooth, Wi-Fi, Bluetooth Low Energy. The apparatus can also transmit using tethered hardware such as a memory stick or wired to receiving hardware such as a handheld tablet or computer. Data may be transmitted directly to the hospital information system (e.g. electronic medical records, laboratory information systems, etc). The data from multiple apparati of the invention may be transmitted to a central host module which in turn communicates with the hospital information systems. The central host module may communicate with the hospital information systems wirelessly (Bluetooth, Wi-Fi, etc) or via a wired connection (LAN, Ethernet, etc). Transmission packages may include patient identifiers, date, time, current measured value, previously measured values, error codes, notes, comments, instrument identifiers, calibration status, etc.

The apparatus can receive information wirelessly using any wireless receiving protocol such as Bluetooth. The apparatus can also receive information using tethered hardware such as a memory stick or wired to receiving hardware such as a handheld tablet or computer. The data may be transmitted to the reusable controller directly from the hospital information systems (e.g. electronic medical records, laboratory information systems, etc). The data from the hospital information systems may be transmitted to a central host module which in turn communicates with multiple reusable controllers. The central host module may communicate with the hospital information systems wirelessly (Bluetooth, Wi-Fi, etc) or via a wired connection (LAN, Ethernet, etc). Transmission packages may include patient identifiers, date, time, modifications to testing protocol, quality control checks, electronic checks, etc.

The information received may be used to modify the testing protocol, perform quality control tests, perform electronic tests, return requested data, verify communication lines are working, etc.

The on board computer of the invention is any well-known microprocessor or application-specific integrated circuit (ASIC) or the like. In an embodiment, the microprocessor or ASIC is commercially-available.

The temperature sensor maybe a commercially available thermocouple chip, or other temperature sensors well-known in the art. Multiple temperature sensors may be housed in the reusable portion as well as the disposable cartridge. The temperature sensor data may be used to modify the execution of a measurement as well as modify a measured value, including temperature regulation, temperature correction, etc.

Flashing or constant signal from a halogen or LED bulb or the like can be used as a visual alarm. The visual alarm may have multiple colors associated with various states of operation and patient condition. The visual alarm may also be displayed as an icon on the display. A piezoelectric buzzer or horn or bell can be used as an audible alarm. The audible alarm may have multiple tones and notes associated with various states of operation and patient condition. The alarm may be housed within the device. The device may communicate with another instrument to trigger an alarm on the instrument.

The electronic display can be an LCD screen, an OLED screen, an LCD touch screen, an OLED touch screen. Discrete buttons on the device may be used to control the electronic display. It will display the necessary information regarding the patient such as patient identifiers, photo ID, time, date, etc. It can also display essential information pertaining to the test(s) that is in progress. It can also serve as a visual alarm when an adverse event is detected. The cardiac marker concentration information will be displayed both numerically and graphically to the doctors so that they can determine whether the patient is undergoing a myocardial infarction. The information can also be further processed to extract information on the actual rise (absolute/relative) in cardiac markers in the patient's blood sample. The display may have a means to lock or password protect or otherwise secure the apparatus, such as, a fingerprint scan or retina scan or facial recognition. Such security prevents tampering or access of information by non-healthcare persons. There can also be a built-in camera or scanner to facilitate fingerprint or retina scan and facial recognition.

The invention will be better understood from the Examples which follow. A person of ordinary skill in the art will readily appreciate that the specific structures and methods discussed in the examples are merely illustrative of the invention as described in the claims, summary of the invention, and the detailed description of the invention.

### EXAMPLES

### Example 1

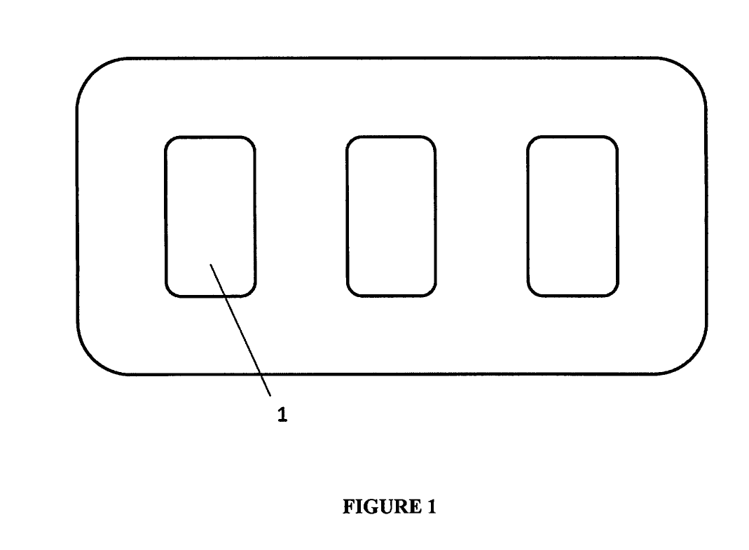

One embodiment of the apparatus of the invention comprises a disposable cartridge. FIG. 1 shows a top view of this embodiment having three individual assay units. Each assay unit [1] has its own piercing element, piercing mechanism, biosensor fluidic circuit, mechanism for generating vacuum or suction, means of moving fluid through the biosensor fluidic circuit, reservoir for collecting waste fluids, chemical reagents, biological reagents, buffers, reaction solutions, and electrodes.

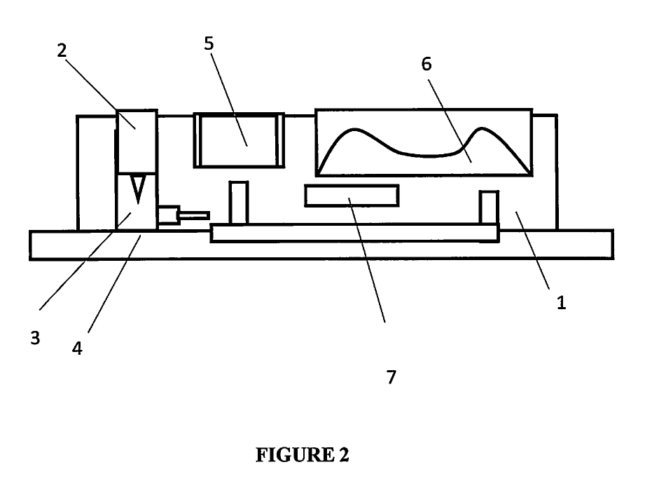

FIG. 2 shows a side view of an individual assay unit [1] within the disposable cartridge. Each individual assay unit has a piercing element [2] and a sampling chamber [3] for obtaining a biological sample at the sampling site [4]. The assay unit has a buffer reservoir [5] and a self-restoring chamber [6] for creating a vacuum and collecting waste solution. The assay unit also comprises an electrochemical sensor [7] for detecting the molecule of interest.

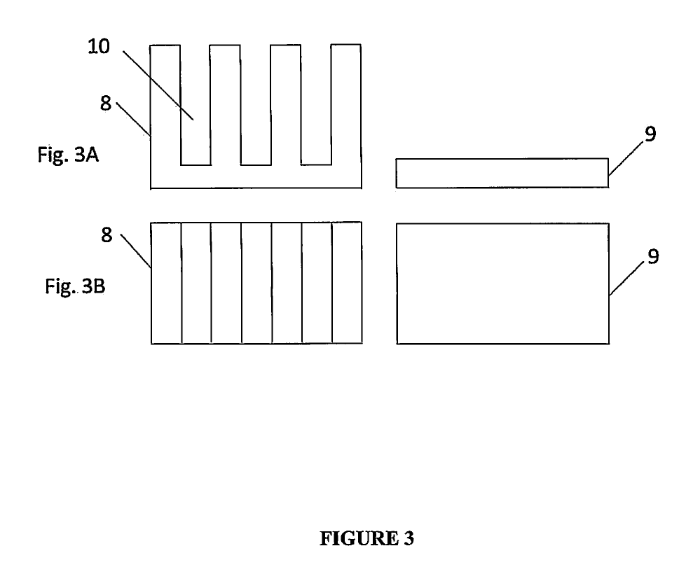

FIG. 3 shows the architecture for the electrode [8] which serves as the electrochemical sensor of the apparatus compared to a conventional silicon electrode [9]. A side view of the electrode is seen in FIG. 3A with the electrode comprising vertical trenches [10] compared to the flat surface of a conventional silicon electrode [9]. FIG. 3B shows a top view of the 3-D sensing electrode of the invention compared to a top view of a conventional silicon electrode. The electrochemical sensor electrode [8] has a significant increase in the surface area of the electrode when compared to conventional silicon electrodes of the same footprint. The larger surface area is made possible by etching a series of vertical trenches into the silicon substrate. Trenches [10] may be created using a number of processes, including but not limited to mechanical dicing, photo patterning and ion etching, etc.

The three dimensional sensing electrodes [8] are silicon-based metal sputtered electrochemical electrodes with larger surface area than conventional silicon electrodes of the same footprint. The increase in surface area may be two-fold to several hundred-fold. The silicon substrate may be sputtered with gold, platinum, or any other appropriate electrically conductive material to create electrodes.

Variability in surface area is minimized due to the precision of the manufacturing processes. Screen printed electrodes are commonly used for electrochemical sensors. However due to the inherent variation in viscosity and ink thickness as well as drying and curing parameters, the effective surface area of screen printed electrodes varies dramatically. By using microfabrication techniques involving sub-micrometer precision photolithography techniques and plasma etch processes as well as thin metal film deposition techniques such as sputtering and evaporation, electrochemical electrodes can be fabricated to sub-micrometer precision.

Advantages of this system include, for example, the lack of blood return to the patient from the system, it's small size and ease of application to patients, it's flexibility for detection of metabolites and/or markers in the patient's blood, automation of the entire process for measuring the marker of interest, and it reduces healthcare worker errors in carrying out the blood testing assays. Another advantage of this embodiment is its ease of application in the field, for example, the system can be attached to a patient and begin monitoring the patient prior to or while a patient is being transported to the hospital.

### Example 2

In use the apparatus is attached to the patient's body preferably via a skin adhesive, patient data is entered into the apparatus, the patient data communicated to the hospital electronic records and an authentication signal is received. On authentication, a spring-loaded skin puncturing needle is released to puncture the skin at a pre-programmed time and the blood sample is allowed to collect at the site of skin penetration until a minimum volume is generated. The blood sample is transported through microfluidic channels into a sensor chamber with an electrochemical-based cardiac marker immunosensor. Immunoassay reagents are automatically introduced into the sensor chamber and the excess blood sample and reagents are collected in a waste chamber. The cardiac marker concentration is measured via an electrochemical signal. The data is made available locally via a display on the apparatus and transmitted to the hospital electronic records. The apparatus uses algorithms which are well known in the art to aid in the diagnosis of a Myocardial Infarction based on the cardiac marker data. The testing may be repeated at a pre-programmed interval or run on an ad-hoc basis.

In use, the sample is introduced into the 3D-Sensing electrodes along the length of the vertical trenches and made to flow along the entire length of the trench. Analytes in the sample interact with chemical, biochemical, and immunological reagents present in the system to produce reaction products that can be measured by the electrodes.

Conventional silicon electrodes are 2-dimensional and the rate of analyte-capture antibody reaction may be limited but the pathlength between any analyte molecule in the bulk fluid and the primary antibody tethered to the 2-D electrode surface. Further, the smaller surface area of a flat electrode limits the number of binding sites available for antibody-analyte interaction. The 3-dimensional construction of the 3D-sensing electrodes reduces the pathlength between any analyte molecule in the bulk fluid and the primary antibody tethered to the 3-D electrode surface. This reduction in pathlength ensures that the immunoassay can be completed in a more rapid manner.

The increased surface area of a 3-dimensional electrode significantly increases the number of antibody binding sites available at the electrode surface for the analyte of interest. The larger number of binding sites increases the resulting sensor signal magnitude for the same analyte concentration, enabling the system to detect smaller amount of the analyte.

### Example 3

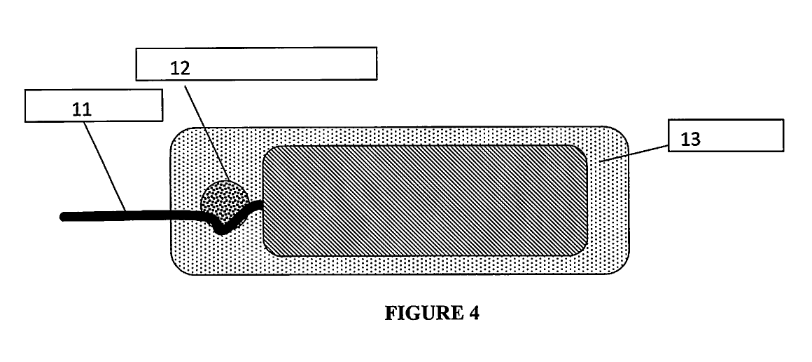

FIG. 4 shows a top view schematic of an alternative embodiment of the disposable cartridge docked onto the reusable portion.

The sampling tubing [11] of the disposable cartridge is wrapped around the cam of the peristaltic pump [12] on the reusable portion [13] to complete the pump. This allows the expensive part of the pump to be reused while the tubing which is in contact with blood is disposable.

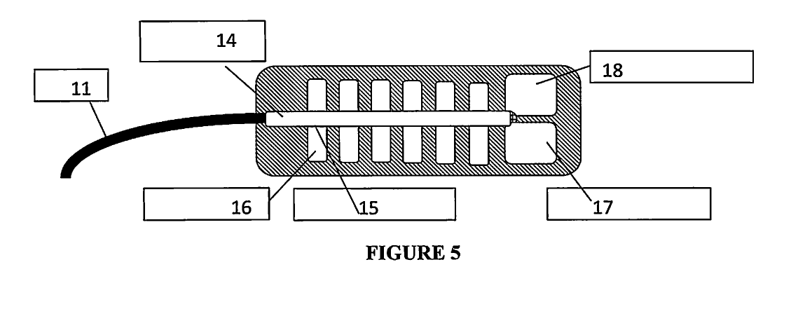

The tubing of the disposable cartridge [11] is in fluidic communication with a common fluidic line [14] within the cartridge as shown in FIG. 5. Along the common fluidic line are individual diaphragm/membrane valves [15] opening to biosensor modules [16]. At the other end of the common fluidic line is a fresh saline reservoir [17] and a waste reservoir [18] and their access are controlled by their respective miniature actuator controlled valves.

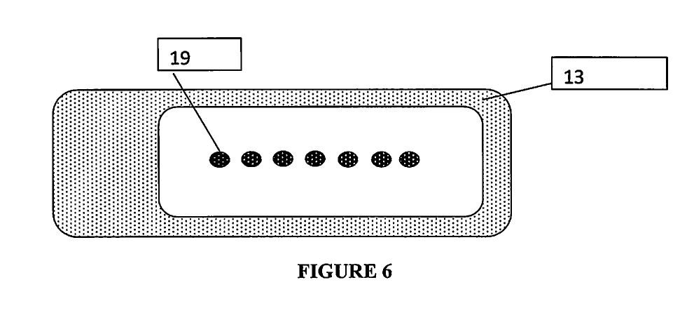

Each individual membrane valve separates biosensor fluidic entry from the common fluidic line. Each individual membrane valve is operated by miniature actuator [19] residing in the reusable portion as shown in FIG. 6.

### Example 4

When in use, the reusable portion is attached to the patient's arm where the blood sample is to be drawn. This ensures close proximity to the arm location where blood sample is to be drawn and this minimizes the dead volume in the tubing between the arm and the disposable cartridge.

After the reusable portion is firmly attached to the patient's arm via various means such as straps or adhesive, the disposable cartridge is docked onto the reusable portion.

Upon docking of the disposable cartridge to the reusable portion, the miniature actuators engages the membrane valves in the disposable cartridge, and the electrical connectors of both disposable cartridge and reusable portion makes contact with each other. The sampling tubing of the disposable cartridge is wrapped around the cam of the peristaltic pump of the reusable portion. The other end of the sampling tubing is attached to the intravenous line that is inserted into the patient's arm.

The sampling tubing and common fluidic line in the disposable cartridge is primed by opening the valve to the waste reservoir and drawing blood up the line and into the waste reservoir. The system may also be primed due to the hydrodynamic head of the circulating blood.

The blood drawn for priming is pushed back by fresh saline buffer into the intravenous line by closing the valve to the waste reservoir and opening valve to the fresh reservoir and reversing the peristaltic pump. This ensures that the entire system is primed without air bubbles and that the sampling line remains unobstructed.

During sampling, the cam of the peristaltic pump rotates and draws the blood sample up the tubing and into the common line of the disposable cartridge. The miniature actuator in the reusable portion energizes and open one of the membrane valve leading to a biosensor fluidic entry into a biosensor fluidic circuit.

The miniature actuator comprises but is not limited to Nitinol based actuators using a wire spring configuration, solenoid based actuator, DC motor based actuator, stepper motor based actuator, servo motor based actuator, electroactive polymer based actuator, MEMS actuator, artificial muscle actuator and piezoelectric actuator.

The biosensor fluidic circuit consists of dried reagent consisting of secondary antibody enzyme conjugate, capture area where the primary antibody is immobilized with respect to the flow channels, electrochemical electrodes and a pouch containing flush buffer with substrate.

The blood sample is draw into the biosensor fluidic circuit and flowed past the dried reagent. The dried reagent dissolved into the blood sample and the secondary antibody enzyme conjugate binds to the target molecule in the blood sample. The blood sample with secondary antibody enzyme conjugate continues toward the capture antibody where the target molecule with secondary antibody enzyme conjugate is captured and tethered to a capture antibody.

The miniature actuator in the reusable portion de-energizes and closes the membrane valve. This seals the blood sample within the individual biosensor fluidic circuit.

The flush buffer and substrate is deployed from the pouch to flush away the blood sample and untethered antibody enzyme conjugate.

The substrate reacts with the enzyme and releases electrochemical product which is measured by the electrochemical electrodes. The measured value is recorded and the corresponding analyte concentration is deduced and transmitted to the hospital electronic record.

The peristaltic pump cam reverses direction and pushes the unused portion of the blood sample in the common line back into the patient while filling the common line with fresh saline buffer to keep the common line unobstructed. In an embodiment, the unused portion of blood sample in the common line is kept uncongealed by introduction of anti-clotting agents. In another separate embodiment, the unused portion of blood sample is flushed into the waste reservoir using a parallel fresh flushing buffer line

The sampling and measurement cycle repeats in accordance to the preprogrammed routine by using a fresh biosensor fluidic circuit within the disposable cartridge for each measurement. At the end of measurement cycle, the disposable cartridge is disposed and the reusable portion is recovered for cleaning and re-use.

### Example 5

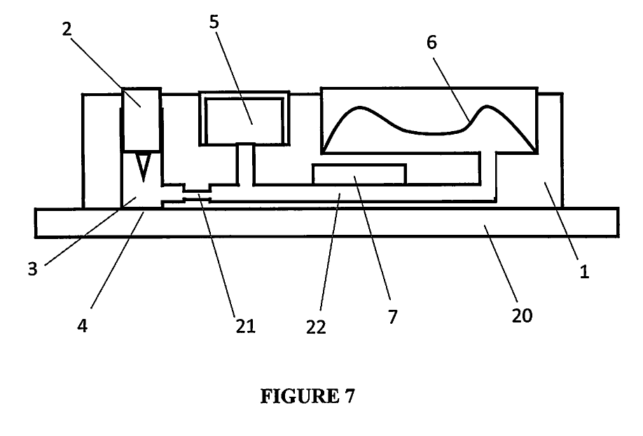

FIG. 7 shows a side view of an individual assay unit [1] within the disposable portion. Each individual assay unit has a piercing element [2] and a sampling chamber [3] for obtaining a biological sample at the sampling site [4] on a patient's skin [20]. The assay unit has a buffer reservoir [5] and a self-restoring chamber [6] for creating a vacuum and collecting waste solution. The liquid in the buffer reservoir can contain chemical reagents needed for detecting the molecule of interest present in the biological sample. An in-line miniature directional flow control valve [21] prevents back flow of buffer and reagents into the sampling chamber [3] during the deployment of liquid stored in the buffer reservoir. The assay unit also comprises an electrochemical sensor [7] residing in a sensing chamber [22] for detecting the molecule of interest.

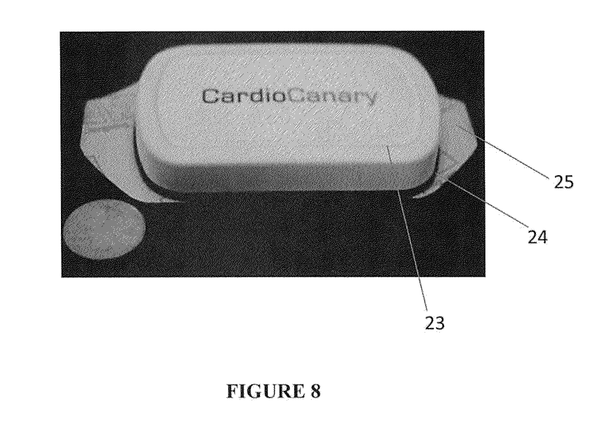

FIG. 8 shows the exterior of an embodiment in a fully assembled state where a reusable portion [23] is attached to the disposable portion [24]. A skin adhesive [25] is present on the underside of the disposable portion. In use, a release liner present on the skin adhesive can be removed to adhere the apparatus to the patient's skin.

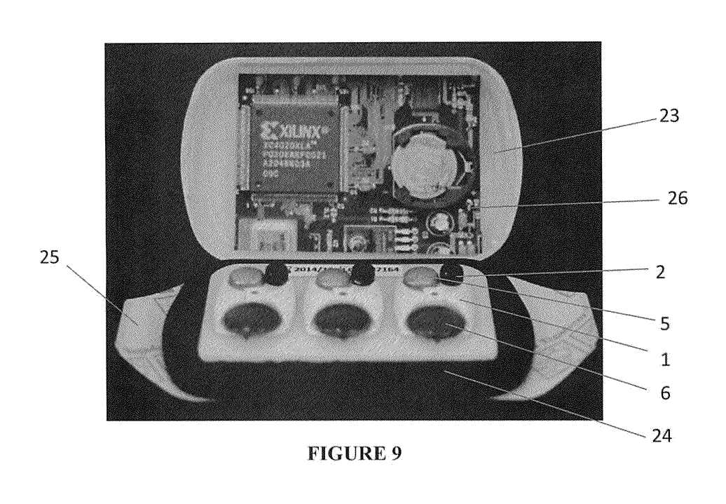

FIG. 9 shows the interior of an embodiment where the reusable portion [23] holds the electronic circuitry along with electromechanical actuators [26]. The reusable portion can communicate with an electronic health record systems as needed to receive signals and transfer data. The disposable portion [24] contains multiple individual assay units [1]. Each assay unit has a piercing element [2], a buffer reservoir [5], and a self-restoring chamber [6] for creating a vacuum and collecting waste solution. The skin adhesive [25] is used to secure the apparatus to the patient's skin.

In use the disposable portion is attached to the patient's body preferably using the skin adhesive. Patient data and testing protocol information is entered or transferred into the reusable portion. Upon activation, a skin puncturing element is released to puncture the stratum corneum at a pre-programmed time and the biological sample is allowed to collect at the site of skin penetration until a minimum sample volume is generated. The sample is transported in a controlled manner through microfluidic channels into a sensor chamber with an electrochemical-based sensor. Electric fields can be used to regulate and control the flow of bodily fluid through the microfluidic channels.

Buffers and other reagents are automatically introduced into the sensor chamber to enable detection of the analyte. Dried chemical and biochemical reagents can be present in microfluidic channels and get mixed into the sample as it flows towards the sensor. Electrochemical products produced by the reaction between the sample and the reagents are measured by the electrochemical electrodes. The measured value is recorded in the reusable portion. This data is made available locally via a display on the reusable portion. It is also transmitted to an electronic health record system as needed.

Once the analyte measurement is completed, the liquids in the sensing chamber are transported and collected within the disposable portion as waste. The sampling and measurement cycle repeats in accordance to the preprogrammed routine in the reusable portion. Different assay units in the disposable portion may be used to measure a single analyte or multiple analytes. Algorithms required to analyze the data and aid in diagnosis can be programmed and resident on the reusable portion. At the end of measurement cycle, the disposable portion is discarded and the reusable portion is recovered for cleaning and re-use.

We claim:

1. A wearable apparatus for diagnosis of a patient, the apparatus comprising:
a biosensor module with an individual spring-loaded skin puncturing element;
an electromechanical actuator to release the spring-loaded skin puncturing element at a programmed time;
a mechanism for moving a sample from a sampling chamber to a sensing chamber;
a mechanism for introducing a buffer and a reagent into the sensing chamber;
a mechanism for collecting a waste product;
a wireless data transceiver capable of receiving an authentication signal and capable of sending a processed biosensor data;
at least one diagnosis algorithm that uses the biosensor data to perform a rapid diagnosis of the patient; and
an onboard computer that controls a release of the skin puncturing element, an operation of the biosensor; the data processing, and the data transmission.

2. The apparatus of claim 1, further comprising at least two additional biosensor modules each with an individual spring-loaded skin puncturing element.

3. The apparatus of claim 1, wherein the biosensor detects a biochemical marker for a disease condition.

4. The apparatus of claim 1, further comprising an electronic display showing a status of a measurement, the biosensor data, and a patient data.

5. The apparatus of claim 3, wherein the housing comprises a means for wireless transmission of a data obtained from the sensor.

6. The apparatus of claim 5, further comprising at least one temperature sensor to compensate for a temperature variation in an electrochemical measurement.

7. The apparatus of claim 6, further comprising at least one microprocessor programmed with a temperature compensation algorithm that uses a temperature data to adjust a calculation of the biosensor data.

8. The apparatus of claim 7, further comprising at least one audible alarm and at least one visual alarm for an adverse event.

9. The apparatus of claim 8, further comprising a skin adhesive wherein the skin adhesive is on the underside of the apparatus between the skin and the apparatus.

10. The apparatus of claim 9, wherein the skin adhesive has cutouts at a location where the skin puncturing element can puncture a skin.

11. The apparatus of claim 5, further comprising at least one motion sensor to determine an orientation of the apparatus.

12. The apparatus of claim 11, wherein the motion sensor is selected from the group consisting of an accelerometer and a gyroscope.

13. A method for diagnosis of a patient, the method comprising the steps of:
contacting a wearable apparatus to a patient's skin, wherein the apparatus comprises an onboard computer, a wireless transceiver, a skin puncturing element, a sample chamber, a biosensor module, and a waste reservoir;
inputting a patient information into the onboard computer;
receiving an authentication signal at the apparatus via the wireless transceiver;
releasing the skin puncturing element at a programmed time to puncture the skin;
collecting a blood sample from the punctured skin in the sample chamber of the apparatus;
transporting the blood sample from the sample chamber into the biosensor module;
deploying a reagent in the biosensor module and routing an excess of the blood sample into the waste reservoir;
measuring a marker concentration in the biosensor module;
transmitting a biosensor data from the apparatus; and
running at least one diagnosis algorithm that uses the biosensor data to perform a rapid diagnosis of the patient.

14. The method of claim 13, wherein the apparatus further comprises a plurality of skin puncturing elements wherein each of the skin puncturing elements is spring loaded.

15. The method of claim 13, wherein the marker is measured electrochemically.

16. The method of claim 13, wherein the biosensor further comprises a optical measuring apparatus, and wherein the marker is measured optically by the optical measuring apparatus.

17. The method of claim 13, wherein the apparatus further comprises at least one additional skin puncturing element.

18. The method of claim 17, wherein the additional skin puncturing elements are released to puncture the skin at predetermined time intervals, and wherein at least one additional blood sample is collected from the additional skin′punctures, transported, analyzed for the marker, and the diagnosis algorithm utilizes the marker data obtained from each blood sample to perform the diagnosis of the patient.

19. An on patient apparatus for diagnosis of a patient, the apparatus comprising:
a biosensor module and a waste reservoir;
an electromechanical mechanism capable of piercing a stratum corneum at a scheduled time;
a mechanism for moving a sample from a sampling chamber to a sensing chamber;
a mechanism for introducing a buffer and a reagent into the sensing chamber;
a wireless data transceiver capable of receiving an authentication signal and capable of sending a processed biosensor data;
at least one diagnosis algorithm that uses the biosensor data to perform a diagnosis of the patient; and
an onboard computer that controls the electromechanical mechanism, an operation of the biosensor; the data processing, and the data transmission.
